(19) United States
(12) Patent Application Publication
(10) Pub. No.: US 2008/0260837 A1
(43) Pub. Date: Oct. 23, 2008

(54) Physically stable aqueous suspensions of active pharmaceuticals

(75) Inventors: Ranga R. Namburi, Robbinsville, NJ (US); Bala Murali K. Vattikuti, West Windsor, NJ (US); Ratna Prabha Mulamalla, Plainsboro, NJ (US); Burgise F. Palkhiwala, Brick, NJ (US)

Correspondence Address: ROBERTS & ROBERTS, LLP;ATTORNEYS AT LAW, P.O. BOX 484, PRINCETON, NJ, 08542-0484, US

(73) Assignee: QPharma, L.L.C.

(21) Appl. No.: 11/788,466

(22) Filed: Apr. 20, 2007

(51) Int. Cl. A61K 9/14; A61K 31/7056; A61K 31/56; A61K 31/55; A61K 31/496; A61K 31/519; A61K 31/522; A61K 31/473; A61K 31/225; A61K 31/135

(52) U.S. Cl. 424/488; 514/43; 514/263.31; 514/262.1; 514/290; 514/548; 514/171; 514/648; 514/254.07; 514/560; 514/217.03; 424/489; 514/471; 514/731

(57) ABSTRACT

The present invention concerns methods of making physically stable aqueous suspensions of sparingly soluble to insoluble in water, active pharmaceuticals. More particularly, the invention provides an aqueous pharmaceutical suspension composition comprising an active pharmaceutical component which is sparingly soluble to insoluble in water; a water soluble, low viscosity grade cellulose polymer with a viscosity range of 3 mPa·s to 50 mPa·s as a surfactant; a suspending agent; and water.

### BACKGROUND OF THE INVENTION

1. Field of the Invention

The present invention concerns methods of making physically stable aqueous suspensions of sparingly soluble to insoluble in water active pharmaceuticals. More particularly, the invention provides physically stable aqueous pharmaceutical suspension composition comprising sparingly soluble to insoluble in water active pharmaceutical component; a water soluble, low viscosity grade cellulose polymer with a viscosity ranging from 3 to 50 mPa·s as a surface active agent; a suspending agent; and water and method of making such suspensions. The suspension shows good content uniformity upon long term storage.

2. Description of the Related Art

Pharmaceuticals which are intended to be orally administered to a patient are known in many dosage forms, including solid forms such as capsules, caplets and tablets, and liquid forms such as solutions, emulsions and suspensions. Children, elderly persons, and persons who are disabled or incapacitated often have trouble swallowing solid dosage forms such as tablets or capsules. For these patients, it is desirable to provide the pharmaceutical in a liquid form. A liquid dosage form is preferable for these patients because of the ease with which it may be swallowed. Also, patients may be more inclined to comply with their medication instruction if the dosages are easier to ingest. However, a common problem associated with liquid pharmaceutical dosage forms is the often disagreeable taste of the drug when in a liquid dosage form. Sometimes, the taste of the drug in the dosage form may be overcome by adding sweeteners or flavoring agents to the liquid dosage which mask the unpleasant taste. Some of the pharmaceutical actives have low solubility in water. To bring them in to solution some vehicles such as propylene glycol, polyethylene glycol, glycerin where in pharmaceutical actives have better solubility could be used in association with water. When such co-solvents are used; masking of the bitter taste of pharmaceuticals requires addition of sweetening agents, bitter masking agents and flavors. However, some times these agents are not totally effective in concealing the unpleasant taste of pharmaceuticals. Hence there is a need to develop suspension dosage forms using water predominantly as vehicle for pharmaceutical actives that have sparingly soluble to insoluble in water characteristics.

Liquid suspension dosage forms also have stability problems associated with maintaining the drugs in suspension. Stability problems include sedimentation, creaming, crystal growth (agglomeration), separation and difficult to re-disperse to obtain original suspensions. Many liquid pharmaceutical suspensions allow the drug to settle out as sediment or creaming to the surface, thereby having variations in the therapeutic concentration of drug in the suspension. This results in under dosing or over dosing of the patient, which may seriously compromise the patient's recovery.

Additionally the pharmaceutical suspension should be readily pourable so that the dose is easy to administer. The requirement that a pharmaceutical suspension be readily pourable effectively places an upper limit on the viscosity of the suspension. This limitation also limits the amount of active pharmaceutical component that the overall composition will suspend.

It would therefore be desirable to develop a ready to use pharmaceutical suspension with a high degree of physical stability, content uniformity and good taste masking characteristics. Therefore, there exists a need for a suspension system for active pharmaceuticals that minimizes sedimentation or creaming of the active ingredients and provides a pleasant tasting liquid dosage. In this regard, U.S. Pat. No. 5,409,907 discloses a pharmaceutical suspension composed of pharmaceutical actives, suspension agents, sweetening agents and flavoring agents, however, no water soluble, low viscosity grade cellulose polymer is used with a surfactant functionality. U.S. Pat. No. 5,374,659 discloses a taste masked pharmaceutical suspension comprising substantially water insoluble pharmaceutical actives, suspension agents and taste masking agents and a process for making such taste masked liquid pharmaceutical suspensions, however, no water soluble, low viscosity grade cellulose polymer is used. U.S. Pat. No. 5,272,137 teaches a pharmaceutical suspension comprising a therapeutic amount of a drug; a suspending system consisting essentially of an effective amount of xanthan gum and microcrystalline cellulose to form a stable suspension system in an aqueous solution; water; and optionally an effective amount of sweetening agents and flavoring agents to provide a palatable taste to said pharmaceutical suspension, however, no water soluble, low viscosity grade cellulose polymer is used. U.S. Pat. No. 4,788,220 teaches a pediatric ibuprofen composition wherein the ibuprofen remains in suspension and the bitter taste of ibuprofen is masked, the primary suspending agents are xanthan gum, microcrystalline cellulose, sodium carboxymelthylcellulose and polysorbate. No water soluble, low viscosity grade cellulose polymer is used. U.S. Pat. No. 5,773,031 discloses a mixture of polymeric coated, sustained release acetaminophen particles and uncoated, quick release acetaminophen particles pressed together in a tablet form. WO03/105804 discloses a semi-solid pharmaceutical suspension for oral administration, comprising a suspension of a water insoluble active ingredient such as ibuprofen in a pharmaceutically acceptable aqueous suspension-stabilizing vehicle. Cellulose derivatives are used; however, no water soluble, low viscosity grade cellulose polymer is used. U.S. Pat. No. 5,759,579 discloses xanthan gum, hydroxyl propyl methyl cellulose and water combination as liquid base suspending agents. However, hydroxyl propyl methyl cellulose of high viscosity grade was used in the combination and not the low viscosity grade cellulose polymer.

The present invention discloses a stable aqueous suspension system for active pharmaceuticals that have sparingly soluble to insoluble in water characteristic, to provide a stable, palatable liquid dosage form. The dosage form comprises of besides the active pharmaceutical ingredient, a water soluble low viscosity grade cellulose polymer with a viscosity range from 3 to 50 mPa·s (measured as a 2 percent solution in water at 20° C.) as a surfactant, a suspending agent, water and other formulation additives. Low viscosity grade cellulose polymers exhibit surface activity and this property allows such a useful benefit of dispersion of solid particles in water. This dosage form is also physico-chemically stable for prolonged periods of storage with good content uniformity and especially well suited for both geriatric and pediatric administration.

### SUMMARY OF THE INVENTION

The invention provides a physically stable pharmaceutical suspension comprising:

a) an active pharmaceutical component which is sparingly soluble to insoluble in water;
b) a water soluble, low viscosity grade cellulose polymer with a viscosity range of from about 3 to about 50 mPa·s, measured at 2.0% concentration in water at 20° C.; as a surfactant
c) a suspending agent; and
d) water.

The invention also provides a method of forming a physically stable pharmaceutical suspension comprising:

I) combining
a) an active pharmaceutical component which is sparingly soluble to insoluble in water, wherein the active pharmaceutical component comprises particle have an average particle size in the range of from about 10 μm to about 200 μm;
b) a water soluble, low viscosity grade cellulose polymer with a viscosity range of from about 3 to about 50 mPa·s, measured at 2.0% concentration in water at 20° C.; as a surfactant
c) a suspending agent; and
d) water;
   II) dispersing the cellulose polymer and suspending agent in a non solvent medium followed by hydration with the water, and then incorporating the active pharmaceutical component with high shear mixing.

### DESCRIPTION OF THE INVENTION

The physically stable pharmaceutical suspension first requires an active pharmaceutical component which is sparingly soluble to insoluble in water. For purposes of this invention, the term “sparingly soluble in water” means 1 part of active pharmaceutical component (solute) requires 30-100 parts of solvent (water) and insoluble in water means 1 part of solute requires at least 10,000 parts of solvent (water) to dissolve. Preferred active pharmaceutical components which are sparingly soluble to insoluble in water non-exclusively include ibuprofen, acetaminophen, guaifenesin, loratadine, propofol, desloratadine, dipyridamole, furosemide, atorvastatin, lovastatin, simvastatin, paroxetin, sertaline, acyclovir, ganicyclovir, itraconazole, ritonavir, saquinavir, beclomethasone dipropionate, flunisolide, fluticasone propionate, budenoside, mometasone furoate, raloxifene HCl. Besides these sparingly soluble to insoluble pharmaceutical actives, other soluble actives could be used in combination and such actives include chlorpheniramine maleate, dextromethorphan HBr, pheniramine maleate, brompheniramine maleate, pseudoephedrine hydrochloride, diphenhydramine hydrochloride, doxylamine succinate, oxycodone. Preferably the active pharmaceutical component which is sparingly soluble to insoluble in water comprises particles have an average particle size in the range of from about 5 μm to about 500 μm, more preferably from about 7.5 μm to about 250 μm and still more preferably from about 10 μm to about 200 μm. The active pharmaceutical component is present in a therapeutic amount, and is preferably present in an amount of from about 0.1 weight percent to about 20 weight percent, more preferably from about 0.5 weight percent to about 15 weight percent, and most preferably from about 1.0 weight percent to about 10.0 weight percent, based on the total volume of the stable pharmaceutical suspension. For the purposes of this invention, the quantity amounts of solid components in the final suspension are given as weight percent based on the final volume of the stable pharmaceutical suspension.

The physically stable pharmaceutical suspension also requires a water soluble, low viscosity grade cellulose polymer with the viscosity range of from about 3 mPa·s to about 50 mPa·s cellulose polymer. For purposes of this invention a water soluble, low viscosity grade cellulose polymer is a polymer which dissolves in water of room temperature by swelling and subsequent hydration to form colloidal solutions having a viscosity of from about 3 mPa·s to about 50 mPa·s, preferably from about 3 to about 15 mPa·s measured as a 2% w/v aqueous solution at 20° C.

Suitable water soluble, low viscosity grade cellulose polymers non-exclusively include hydroxypropyl methyl cellulose (hypromellose), hydroxyethyl cellulose, hydroxypropyl cellulose, and combinations thereof. Hydroxypropyl methylcellulose polymers are preferred. These low viscosity grade cellulose polymers exhibit surface activity for dispersion of solid particles in water.

Hydroxypropyl methylcellulose polymers which may be used in the present invention are available under the brand name Methocel™ available from Dow Chemical Co. Examples of hydroxypropyl methylcellulose polymers of a low viscosity grade include those available under the brand names Methocel E3 Prem.LV, Methocel E5 Prem.LV, Methocel E6 Prem.LV, Methocel E-15 Prem.LV, and Methocel E50 Prem.LV whose 2% by weight aqueous solutions have viscosities of 3 mPa·s, 5 mPa·s, 6 mPa·s, 15 mPa·s, and 50 mPa·s respectively. The hydroxypropyl methyl cellulose polymers that may be used in the present invention include, for example, polymers available under the brand name Pharmacoat from ShinEtsu, Spectracel and Tylopur. Other cellulose polymers include hydroxy ethyl cellulose, hydroxy ethyl methyl cellulose, and hydroxy propyl cellulose under different brand names.

The water soluble, low viscosity grade cellulose polymer with a viscosity range of 3 mPa·s to 50 mPa·s component is preferably present in an amount of from about 0.1 weight percent to about 5.0 weight percent, more preferably from about 0.5 weight percent to about 2.0 weight percent, and most preferably from about 0.75 weight percent to about 1.5 weight percent, based on the total volume of the physically stable pharmaceutical suspension.

The stable pharmaceutical suspension also requires a suspending or viscosity increasing agent. Suitable suspending or viscosity increasing agents non-exclusively include high viscosity grade polymers (viscosities resulting in more than 50 mPa·s of 1.0% aqueous solution at room temperature 20° C.). Such may include cellulosic polymers, polysaccharide polymers and acrylic acid derivatives, gums and others. Examples include xanthan gum (a high molecular weight polysaccharide, with a viscosity of 1200-1600 mPa·s when measured on 1.0 percent salt solution; xanthan gum is available from several commercial suppliers such as CP Kelco under the brand name Xanthural, Rhodia under the brand name Rhodigel), carrageenan (a polysaccharide, material under the brand name Genuvisco from CP Kelco is useful for present invention), carboxymethylcellulose calcium, carboxymethylcellulose sodium, methylcellulose, carboxymethylcellulose sodium 12, hydroxyethyl cellulose, hydroxypropyl cellulose, hydroxypropyl methylcellulose (hypromellose), microcrystalline cellulose. Other suitable suspending or viscosity increasing agents non-exclusively include acacia, agar, alginic acid, aluminum monostearate, attapulgite-activated, attapulgite, colloidal activated bentonite, bentonite-purified, bentonite magma, carbomers, dextrin, gelatin, gellan gum, guar gum, magnesium aluminum silicate, maltodextrin, pectin, polyethylene oxide, polyvinyl alcohol, povidone, propylene glycol alginate, silicon dioxide, silicon dioxide-colloidal, sodium alginate, starch, corn starch, potato starch, tapioca, wheat tragacanth, as well as combinations of suspending or viscosity increasing agent. The suspending or viscosity increasing agent is preferably present in an amount of from about 0.05 weight percent to about 5.0 weight percent, more preferably from about 0.1 weight percent to about 1.5 weight percent, and most preferably from about 0.2 weight percent to about 0.6 weight percent, based on the total volume of the physically stable pharmaceutical suspension.

The physically stable pharmaceutical suspension may optionally include other wetting agents or surface active agents. Suitable wetting agents non-exclusively include benzalkonium chloride, benzethonium chloride, cetylpyridinium chloride, docusate sodium, nonoxynol 9, octoxynol 9, poloxamer, polyoxyl 10 oleyl ether, polyoxyl 20 cetostearyl ether, polyoxyl 35 castor oil, polyoxyl 40 hydrogenated castor oil, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, sorbitan sesquioleate, sorbitan trioleate, tyloxapol and combinations thereof. When a wetting agent is included it is preferably present in an amount of from about 0.05 weight percent to about 1.0 weight percent, more preferably from about 0.1 weight percent to about 0.5 weight percent, and most preferably from about 0.1 weight percent to about 0.2 weight percent, based on the total volume of the stable pharmaceutical suspension.

The physically stable pharmaceutical suspension may optionally include a sweetening agent. Suitable sweetening agents non-exclusively include acesulfame potassium, aspartame, acesulfame, dextrates, dextrose, fructose, high fructose corn syrup, galactose, maltitol, maltose, mannitol, saccharin, saccharin calcium, saccharin sodium, sorbitol, sorbitol solution, sucralose, sucrose, confectioner's syrup, tagatose, and combinations thereof. When a sweetening agent is included it is preferably present in an amount of from about 0.05 weight percent to about 60.0 weight percent, more preferably from about 0.1 weight percent to about 30.0 weight percent, and most preferably from about 0.2 weight percent to about 2.0 weight percent, based on the total volume of the stable pharmaceutical suspension.

The physically stable pharmaceutical suspension may optionally include an antimicrobial preservative. Suitable antimicrobial preservatives non-exclusively include benzalkonium chloride, benzalkonium chloride solution, benzethonium chloride, benzoic acid, benzyl alcohol, butylparaben, cetrimonium bromide, cetylpyridinium chloride, chlorobutanol, chlorocresol, cresol, ethylparaben, methylparaben, methylparaben sodium, phenol, phenoxyethanol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol and combinations thereof. When an antimicrobial preservative is included it is preferably present in an amount of from about 0.05 weight percent to about 2.0 weight percent, more preferably from about 0.1 weight percent to about 1.0 weight percent, and most preferably from about 0.2 weight percent to about 0.5 weight percent, based on the total volume of the stable pharmaceutical suspension.

The physically stable pharmaceutical suspension may optionally include a flavor or perfume. Suitable flavors and perfumes non-exclusively include grape, cherry, berry, bubble gum and combinations thereof. When a flavor or perfume is included it is preferably present in an amount of from about 0.05 weight percent to about 2.0 weight percent, more preferably from about 0.1 weight percent to about 1.0 weight percent, and most preferably from about 0.1 weight percent to about 0.2 weight percent, based on the total volume of the stable pharmaceutical suspension.

The physically stable pharmaceutical suspension may optionally include an acidifying agent. Suitable acidifying agents non-exclusively include acetic acid, acetic acid-glacial, citric acid-anhydrous, citric acid monohydrate, fumaric acid, hydrochloric acid, hydrochloric acid-diluted, malic acid, nitric acid, phosphoric acid, phosphoric acid-diluted, propionic acid, sulfuric acid, tartaric acid and combinations thereof. When an acidifying agent is included it is preferably present in an amount of from about 0.005 weight percent to about 1.0 weight percent, more preferably from about 0.01 weight percent to about 0.5 weight percent, and most preferably from about 0.1 weight percent to about 0.2 weight percent, based on the total volume of the stable pharmaceutical suspension.

The physically stable pharmaceutical suspension may optionally include an alkalizing agent. Suitable alkalizing agents non-exclusively include ammonia solution-strong, ammonium carbonate, diethanolamine, potassium hydroxide, sodium bicarbonate, sodium borate, sodium carbonate, sodium hydroxide, trolamine and combinations thereof. When an alkalizing agent is included it is preferably present in an amount of from about 0.005 weight percent to about 1.0 weight percent, more preferably from about 0.01 weight percent to about 0.5 weight percent, and most preferably from about 0.1 weight percent to about 0.2 weight percent, based on the total volume of the stable pharmaceutical suspension.

The physically stable pharmaceutical suspension may optionally include a colorant. Suitable coloring agents non-exclusively include FD&C Blue No. 1. FD&C Blue No. 2, FD&C Green No. 3, FD&C Red No. 4, FD&C Yellow No. 5, FD&C Yellow No. 6, D&C Blue No. 4, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 7, and combinations thereof. When a colorant is included it is preferably present in an amount of from about 0.0003 weight percent to about 0.01 weight percent, more preferably from about 0.0005 weight percent to about 0.005 weight percent, and most preferably from about 0.001 weight percent to about 0.02 weight percent, based on the total volume of the stable pharmaceutical suspension.

The physically stable pharmaceutical suspensions may be formed by dispersion of water soluble low viscosity grade cellulose polymer and suspending agent in non-solvent medium such as glycerine (a component of the composition) by formation of fluid slurry and then adding them in to main vehicle water to obtain complete hydration of polymers. A non-solvent medium is a medium which lacks water and hence a medium in which the cellulose polymer does not undergo hydration. Other non-solvent media non-exclusively include propylene glycol and polyethylene glycol. After combining of all the components prior to the addition of flavor, active pharmaceutical ingredient is dispersed through high shear mixing operation of homogenization or triclover blending operation or any other suitable high shear mixing operation. The flavor components are included at the end of the process of combining the composition. Finally the volume was made up and the suspension was allowed overnight to de-aerate prior to filling in to bottles.

Preferably the physically stable pharmaceutical suspension has a viscosity range of 1000 mPa·s to 2500 mPa·s when measured at 20° C.

### EXAMPLES

The invention will now be illustrated by examples. Preferred method of combining the composition ingredients is also outlined in examples.

### Example 1

Ibuprofen Suspension Pediatric Liquid Dosage Form:

This example discloses a pharmaceutical suspension containing Ibuprofen and a process for manufacturing this suspension. The ingredients contained in the Ibuprofen suspension are as follows.

| | | Weight in |
|---|---|---|
| Ingredient | Percentage | Grams |
| Ibuprofen | 2.000 | 20.00 |
| Sucralose | 0.050 | 0.50 |
| Citric Acid (Anhydrous) | 0.200 | 2.00 |
| Glycerin | 10.000 | 100.00 |
| Sorbitol 70% solution | 5.000 | 50.00 |
| High Fructose Corn Syrup | 47.500 | 475.00 |
| 55 grade | | |
| Xanthan Gum | 0.260 | 2.60 |
| Hydroxy propyl methyl | 1.000 | 10.00 |
| cellulose 6 mPa.s grade | | |
| Methyl paraben | 0.150 | 1.50 |
| Propyl paraben | 0.050 | 0.50 |
| Sodium Hydroxide | 0.060 | 0.60 |
| FD&C Red #40 | 0.001 | 0.01 |
| Bubble Gum SN 578840 | 0.150 | 1.50 |
| Purified Water USP | qs | to 1000.0 mL |

### Processing Directions:

The Ibuprofen suspension was prepared as follows:
1. Take High Fructose Corn syrup and purified water 300 g in to a tared container.
2. In a separate container take glycerin 50 g and disperse xanthan gum under mixing. Add this to the main bulk under stirring.
3. In a separate container take glycerin 50 g and disperse hydroxypropyl methylcellulose 6 mPa·s grade and add it to the main bulk under stirring.
4. Add and dissolve Citric Acid and Sucralose separately in purified water and add to the main bulk under continued stirring
5. Dissolve parabens in aqueous sodium hydroxide solution and add to the main bulk under stirring.
6. Add sotbitol 70% solution to the main bulk under stirring
7. Disperse Ibuprofen in main bulk using high shear mixer (Homogenizer)
8. Incorporate colors dissolved in water and add to the main bulk under stirring. Add flavors to the main bulk directly under continued stirring.
9. Make up the volume, store overnight to de-aerate and fill in to bottles.

### Example 2

### Acetaminophen Suspension Pediatric Liquid Dosage Form:

This example discloses a pharmaceutical suspension containing Acetaminophen and a process for manufacturing this suspension. The ingredients contained in the Acetaminophen suspension are as follows.

| | | Weight in |
|---|---|---|
| Ingredient | Percentage | Grams |
| Acetaminophen | 3.200 | 32.00 |
| Sorbitol 70% solution | 16.600 | 166.00 |
| Citric Acid | 0.125 | 1.25 |
| (Anhydrous) | | |
| Glycerin | 30.000 | 300.00 |
| Propylene glycol | 5.000 | 50.00 |
| Sodium Benzoate | 0.250 | 2.50 |
| Hydroxypropyl | 1.000 | 10.00 |
| methylcellulose | | |
| 6 mPa · s grade | | |
| Butyl paraben | 0.025 | 0.25 |
| Xanthan gum | 0.250 | 2.50 |
| High fructose corn | 42.000 | 420.00 |
| syrup 55 | | |
| Sucralose | 0.100 | 1.00 |
| Bubble gum flavor | 0.600 | 6.00 |
| SN 281975 | | |
| FD&C Red No. 40 | 0.0017 | 0.017 |
| D&C Red No. 33 | 0.00033 | 0.0033 |
| Purified Water USP | qs | to 1000.0 mL |

### Processing Directions:

The Acetaminophen suspension was prepared as follows:
1. Take High Fructose Corn syrup and 150 g of water in to a tared container.
2. In a separate container take glycerin 150 g and disperse xanthan gum under mixing. Add this to the main bulk under stirring.
3. Dissolve butyl paraben in propylene glycol and add to the main bulk under stirring.
4. Add and dissolve Citric Acid, Sucralose and Sodium benzoate separately in water and add to the main bulk under continued stirring.
5. In a separate container take glycerin 150 g and disperse hydroxypropyl methylcellulose 6 mPa·s grade and add it to the main bulk under stirring.
6. Add sotbitol 70% solution to the main bulk under stirring.
7. Disperse Acetaminophen in main bulk using high shear mixer (Homogenizer)
8. Incorporate colors dissolved in water and add to the main bulk under stirring. Add flavors to the main bulk directly under continued stirring.
9. Make up the volume, store overnight to de-aerate and fill in to bottles.

### Example 3

Acetaminophen, Chlorpheniramine maleate and Dextromethorphan HBr Suspension Pediatric Liquid Dosage Form:

This example discloses a pharmaceutical suspension containing Acetaminophen, Chlorphenaramine maleate and Dextromethorphan HBr and a process for manufacturing this suspension. Chlorpheniramine maleate and Dextromethorphan HBr are in solution form and Acetaminophen is in suspended form. The ingredients contained in the suspension are as follows.

| | | Weight in |
|---|---|---|
| Ingredient | Percentage | Grams |
| Acetaminophen | 3.200 | 32.00 |
| Dextromethorphan | 0.100 | 1.000 |
| HBr | | |
| Chlorpheneramine | 0.020 | 0.200 |
| maleate | | |
| Sorbitol 70% solution | 16.600 | 166.00 |
| Citric Acid | 0.125 | 1.25 |
| (Anhydrous) | | |
| Glycerin | 30.000 | 300.00 |
| Propylene glycol | 5.000 | 50.00 |
| Sodium Benzoate | 0.250 | 2.50 |
| Hydroxypropyl | 1.000 | 10.00 |
| methylcellulose | | |
| 6 mPa · s grade | | |
| Butyl paraben | 0.025 | 0.25 |
| Xanthan gum | 0.250 | 2.50 |
| High fructose corn | 42.000 | 420.00 |
| syrup 55 | | |
| Sucralose | 0.100 | 1.00 |
| Bubble gum | 0.600 | 6.00 |
| SN281975 | | |
| FD&C Red No. 40 | 0.0017 | 0.017 |
| D&C Red No. 33 | 0.00033 | 0.0033 |
| Purified Water USP | qs | to 1000.0 mL |

### Processing Directions:

The Acetaminophen, Chlorpheniramine maleate and Dextromethorphan HBr suspension was prepared as follows:
1. Take High Fructose Corn syrup and 75 g of purified water in to a tared container.
2. In a separate container take glycerin 100 g and disperse xanthan gum under mixing. Add this to the main bulk under stirring.
3. Dissolve butyl paraben in propylene glycol and add to the main bulk under stirring.
4. Add and dissolve Citric Acid, Sucralose and Sodium benzoate separately in water and add to the main bulk under continued stirring
5. In a separate container take glycerin 150 g and disperse hydroxypropyl methylcellulose and add it to the main bulk under stirring.
6. Add sotbitol 70% solution to the main bulk under stirring
7. Disperse Acetaminophen in main bulk using high shear mixer (Homogenizer)
8. In a separate container take glycerin 50 g add and dissolve Dextromethorphan HBr and add to the main bulk under stirring.
9. Add and Dissolve Chlorpheniramine maleate in purified water and add to the main bulk under stirring.
10. Incorporate colors dissolved in water and add to the main bulk under stirring. Add flavors to the main bulk directly under continued stirring.
11. Make up the volume, store overnight to de-aerate and fill in to bottles.

### Example 4

### Ibuprofen Suspension Liquid Dosage Form for Infants:

This example discloses a suspension containing concentrated Ibuprofen for infants and a process for manufacturing this suspension. The ingredients contained in the Ibuprofen suspension are as follows.

| | | Weight in |
|---|---|---|
| Ingredient | Percentage | Grams |
| Ibuprofen | 4.000 | 40.00 |
| Sucralose | 0.050 | 0.50 |
| Citric Acid | 0.200 | 2.00 |
| (Anhydrous) | | |
| Glycerin | 5.000 | 50.00 |
| Sorbitol 70% | 5.000 | 50.00 |
| Solution | | |
| Polysorbate 80 | 0.200 | 2.00 |
| High Fructose Corn | 47.500 | 475.00 |
| Syrup 55 grade | | |
| Xanthan Gum | 0.300 | 3.00 |
| Hydroxypropyl | 1.000 | 10.00 |
| methylcellulose | | |
| 6 mPa · s grade | | |
| Maltodextrin M 150 | 5.000 | 50.00 |
| Methyl paraben | 0.150 | 1.50 |
| Propyl paraben | 0.050 | 0.50 |
| Sodium Hydroxide | 0.060 | 0.60 |
| FD&C Red No. 40 | 0.001 | 0.01 |
| Bubble Gum | 0.150 | 1.50 |
| SN578840 | | |
| Purified Water USP | qs | to 1000.0 mL |

### Processing Directions:

Follow the processing directions as under Example 1. This example additionally contains polysorbate 80 and maltodextrin ingredients. Prior to the addition of sorbitol step as in Example 1, incorporate maltodextrin dissolved in water and polysorbate 80 in to main bulk under continued mixing.

### Example 5

### Guaifenesin Suspension Liquid Dosage Form:

This example discloses a pharmaceutical suspension containing Guaifenesin and a process for manufacturing this suspension. The ingredients contained in the Guaifenesin suspension are as follows.

| | | Weight in |
|---|---|---|
| Ingredient | Percentage | Grams |
| Guaifenesin | 2.000 | 20.00 |
| Sucralose | 0.150 | 1.50 |
| Citric Acid | 0.100 | 1.00 |
| (Anhydrous) | | |
| Glycerin | 6.000 | 60.00 |
| Sorbitol 70% | 90.000 | 900.00 |
| Solution | | |
| Xanthan Gum | 0.250 | 2.50 |
| Hydroxypropyl | 0.100 | 1.00 |
| methylcellulose | | |
| 6 mPa · s grade | | |
| Propylene Glycol | 5.000 | 50.00 |
| Methyl paraben | 0.150 | 1.50 |
| Propyl paraben | 0.050 | 0.50 |
| Sodium saccharin | 0.200 | 2.00 |
| FD&C Red No. 40 | 0.0025 | 0.025 |
| D&C Red No. 33 | 0.00075 | 0.0075 |
| Wild Berry | 0.200 | 2.00 |
| SN583357 | | |
| Purified Water USP | qs | to 1000.0 mL |

### Processing Directions:

1. The Guaifenesin suspension was prepared as follows:
2. Add and disperse Guaifenesin in Sorbitol 70% solution 900 g in a tared container.
3. In a separate container take glycerin 50 g and disperse xanthan gum under mixing. Add this to the above bulk under stirring.
4. In a separate container take glycerin 10 g and disperse Hydroxypropyl methylcellulose 6 mPa·s grade under mixing. Add this to the main bulk under stirring.
5. Add and dissolve parabens in Propylene glycol and add to the main bulk under stirring.
6. Add and dissolve Citric Acid, Sucralose and Sodium saccharine separately in water and add to the main bulk under stirring.
7. Incorporate colors dissolved in water and add to the main bulk under stirring. Add flavors to the main bulk directly under continued stirring.
8. Make up the volume, store overnight to de-aerate and fill in to bottles.

### Example 6

### Acetaminophen Suspension Pediatric Liquid Dosage Form:

This example discloses a pharmaceutical suspension containing Acetaminophen and a process for manufacturing this suspension. This example uses carrageenan gum in place of xanthan gum and an additional suspending agent carboxymethyl cellulose and microcrystalline cellulose. The ingredients contained in the Acetaminophen suspension are as follows.

| | | Weight in |
|---|---|---|
| Ingredient | Percentage | Grams |
| Acetaminophen | 3.200 | 32.00 |
| Sorbitol 70% solution | 16.600 | 166.00 |
| Citric Acid | 0.125 | 1.25 |
| (Anhydrous) | | |
| Glycerin | 45.000 | 450.00 |
| Propylene glycol | 5.000 | 50.00 |
| Sodium Benzoate | 0.250 | 2.50 |
| Hydroxypropyl | 1.000 | 10.00 |
| methylcellulose | | |
| 6 mPas grade | | |
| Microcrystalline | 0.450 | 4.50 |
| cellulose and carboxy | | |
| methylcellulose | | |
| sodium (RC 591) | | |
| Butyl paraben | 0.025 | 0.25 |
| Carrageenan Gum | 0.225 | 2.25 |
| High fructose corn | 42.000 | 420.00 |
| syrup 55 | | |
| Sucralose | 0.100 | 1.00 |
| Bubble gum SN | 0.600 | 6.00 |
| 578840 | | |
| FD&C Red No. 40 | 0.0017 | 0.017 |
| D&C Red No. 33 | 0.00033 | 0.0033 |
| Purified Water USP | qs | to 1000.0 mL |

### Processing Directions:

The Acetaminophen suspension was prepared as follows:
1. Take High Fructose Corn syrup in to a tared container.
2. In a separate container take glycerin 225 g and disperse Carrageenan gum under mixing. Add this to the main bulk under stirring.
3. Dissolve Butyl paraben in propylene glycol and disperse Microcrystalline cellulose and carboxy methylcellulose sodium (RC 591) and add to the main bulk under stirring.
4. Add and dissolve Citric Acid, Sucralose and Sodium benzoate separately in water and add to the main bulk under continued stirring
5. In a separate container take glycerin 225 g and disperse hydroxypropyl methylcellulose 6 mPa s grade and add it to the main bulk under stirring.
6. Add sotbitol 70% solution to the main bulk under stirring.
7. Disperse Acetaminophen in main bulk using high shear mixer (Homogenizer)
8. Incorporate colors dissolved in water and add to the main bulk under stirring. Add flavors to the main bulk directly under continued stirring.
9. Make up the volume, store overnight to de-aerate and fill in to bottles.

### Example 7

### Concentrated Acetaminophen Suspension Infant Drops Liquid Dosage Form:

This example discloses a pharmaceutical suspension containing Acetaminophen at 10.0 percent concentration and a process for manufacturing this suspension for Infants. The ingredients contained in the Acetaminophen Infants suspension are as follows.

| | | Weight in |
|---|---|---|
| Ingredient | Percentage | Grams |
| Acetaminophen | 10.000 | 100.00 |
| Sorbitol 70% solution | 16.600 | 166.00 |
| Citric Acid | 0.125 | 1.25 |
| (Anhydrous) | | |
| Glycerin | 30.000 | 300.00 |
| Sodium Benzoate | 0.250 | 2.50 |
| Hydroxypropyl | 1.000 | 10.00 |
| methylcellulose | | |
| 6 mPas grade | | |
| Xanthan Gum | 0.250 | 2.50 |
| High fructose corn | 42.000 | 420.00 |
| syrup 55 | | |
| Sucralose | 0.100 | 1.00 |
| Bubble gum SN | 0.600 | 6.00 |
| 58840 | | |
| FD&C Red No. 40 | 0.0017 | 0.017 |
| D&C Red No. 33 | 0.00033 | 0.0033 |
| Purified Water USP | qs | to 1000.0 mL |

### Processing Directions:

The Acetaminophen Infants suspension was prepared as follows:
1. Take High Fructose Corn syrup and 125 g purified water in to a tared container.
2. In a separate container take glycerin 150 g and disperse Xanthan gum under mixing. Add this to the main bulk under stirring.
3. Add and dissolve Citric Acid, Sucralose and Sodium benzoate separately in water and add to this to the main bulk under continued stirring
4. In a separate container take glycerin 150 g and disperse hydroxypropyl methylcellulose 6 mPa s grade and add it to the main bulk under stirring.
5. Add sorbitol 70% solution to the main bulk under stirring.
6. Disperse Acetaminophen in main bulk using high shear mixer (Homogenizer)
7. Incorporate colors dissolved in water and add to the main bulk under stirring. Add flavors to the main bulk directly under continued stirring.
8. Make up the volume, store overnight to de-aerate and fill in to bottles.

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

What is claimed is:

1. A physically stable pharmaceutical suspension comprising:
a) an active pharmaceutical component which is sparingly soluble to insoluble in water;
b) a water soluble, low viscosity grade cellulose polymer with a viscosity range of from about 3 to about 50 mPa·s, measured at 2.0% concentration in water at 20° C. as a surfactant;
c) a suspending agent; and
d) water.

2. The physically stable pharmaceutical suspension of claim 1 wherein the active pharmaceutical component comprises particles have an average particle size in the range of from about 10 μm to about 200 μm.

3. The physically stable pharmaceutical suspension of claim 1 wherein the active pharmaceutical component which is sparingly soluble to insoluble in water comprises ibuprofen, acetaminophen, guaifenesin, loratadine, propofol, desloratadine, dipyridamole, furosemide, atorvastatin, lovastatin, simvastatin, paroxetin, sertaline, acyclovir, ganicyclovir, itraconazole, ritonavir, saquinavir, beclomethasone dipropionate, flunisolide, fluticasone propionate, budenoside, mometasone furoate, raloxifene HCl, and combinations thereof.

4. The physically stable pharmaceutical suspension of claim 1 further comprising a water soluble active pharmaceutical component comprising
chlorpheniramine maleate, dextromethorphan HBr, pheniramine maleate, bromphenramine maleate, pseudoephedrine hydrochloride, diphenhydramine hydrochloride, doxylamine succinate, oxycodone and combination thereof.

5. The physically stable pharmaceutical suspension of claim 1 wherein the water soluble, low viscosity grade cellulose polymer is a polymer which forms liquid colloidal solutions having a viscosity of from about 3 mPa·s to about 15 mPa·s when measured on a 2% w/v aqueous solution at 20° C.

6. The physically stable pharmaceutical suspension of claim 1 wherein the water soluble, low viscosity grade cellulose polymer comprises hydroxypropyl methyl cellulose, hydroxyethyl cellulose, hydroxypropyl cellulose, methyl cellulose, and combinations thereof.

7. The physically stable pharmaceutical suspension of claim 1 wherein the water soluble, low viscosity grade cellulose polymer comprises hydroxypropyl methyl cellulose which forms liquid colloidal solutions having a viscosity of about 6 mPa·s when measured on a 2% w/v aqueous solution at 20° C.

8. The physically stable pharmaceutical suspension of claim 1 wherein the suspending agent comprises a water soluble, high viscosity grade cellulose, a polysaccharide, a gum, an acrylic acid derivative or combinations thereof which forms a liquid colloidal solution has a viscosity of from in excess of 50 mPa·s in a 1% w/v aqueous solution.

9. The physically stable pharmaceutical suspension of claim 1 wherein the suspending agent comprises xanthan gum, carrageenan gum, carboxymethylcellulose calcium, carboxymethylcellulose sodium, methylcellulose, carboxymethylcellulose sodium 12, hydroxyethyl cellulose, hydroxypropyl cellulose, hydroxypropyl methylcellulose (hypromellose), microcrystalline cellulose, acacia, agar, alamic acid, alginic acid, aluminum monostearate, attapulgite-activated, attapulgite, colloidal activated bentonite, bentonite-purified, bentonite magma, carbomer polymer, cellulose, dextrin, gelatin, gellan gum, guar gum, magnesium aluminum silicate, maltodextrin, pectin, polyethylene oxide, polyvinyl alcohol, povidone, propylene glycol alginate, silicon dioxide, silicon dioxide-colloidal, sodium alginate, starch, corn starch, potato starch, tapioca, wheat tragacanth, or combinations thereof.

10. The physically stable pharmaceutical suspension of claim 1 which further comprises an additional wetting agent.

11. The physically stable pharmaceutical suspension of claim 1 which further comprises an additional wetting agent comprising at least one of benzalkonium chloride, benzethonium chloride, cetylpyridinium chloride, docusate sodium, nonoxynol 9, octoxynol 9, poloxamer, polyoxyl 10 oleyl ether, polyoxyl 20 cetostearyl ether, polyoxyl 35 castor oil, polyoxyl 40 hydrogenated castor oil, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, sorbitan sesquioleate, sorbitan trioleate, and tyloxapol or combinations thereof.

12. The physically stable pharmaceutical suspension of claim 1 which further comprises a sweetening agent.

13. The physically stable pharmaceutical suspension of claim 1 which further comprises a sweetening agent which comprises at least one of acesulfame potassium, aspartame, acesulfame, dextrates, dextrose, dextrose excipient, fructose, galactose, maltitol, maltose, mannitol, saccharin, saccharin calcium, saccharin sodium, sorbitol, sorbitol solution, sucralose, sucrose, sugar, sugar-compressible, high fructose corn syrup, confectioner's syrup, and tagatose.

14. The physically stable pharmaceutical suspension of claim 1 which further comprises an antimicrobial preservative.

15. The physically stable pharmaceutical suspension of claim 1 which further comprises an antimicrobial preservative which comprises at least one of benzalkonium chloride, benzalkonium chloride solution, benzethonium chloride, benzoic acid, benzyl alcohol, butylparaben, cetrimonium bromide, cetylpyridinium chloride, chlorobutanol, chlorocresol, cresol, ethylparaben, methylparaben, methylparaben sodium, phenol, phenoxyethanol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, and thymol.

16. The physically stable pharmaceutical suspension of claim 1 which further comprises at least one flavor, at least one perfume, or at least one flavor plus at least one perfume.

17. The physically stable pharmaceutical suspension of claim 1 which further comprises a flavor agent comprising at least one of grape, cherry, bubble gum, berry, or combinations thereof.

18. The physically stable pharmaceutical suspension of claim 1 which further comprises an acidifying agent.

19. The physically stable pharmaceutical suspension of claim 1 which further comprises an acidifying agent comprising at least one of acetic acid, acetic acid-glacial, citric acid-anhydrous, citric acid monohydrate, fumaric acid, hydrochloric acid, hydrochloric acid-diluted, malic acid, nitric acid, phosphoric acid, phosphoric acid-diluted, propionic acid, sulfuric acid and tartaric acid.

20. The physically stable pharmaceutical suspension of claim 1 which further comprises an alkalizing agent comprising at least one of an ammonia solution, ammonium carbonate, diethanolamine, potassium hydroxide, sodium bicarbonate, sodium borate, sodium carbonate, sodium hydroxide, and trolamine.

21. The physically stable pharmaceutical suspension of claim 1 which further comprises a colorant.

22. The physically stable pharmaceutical suspension of claim 1 which further comprises a colorant comprising at least one of FD&C Blue No. 1. FD&C Blue No. 2, FD&C Green No. 3, FD&C Red No. 4, FD&C Yellow No. 5, FD&C Yellow No. 6, D&C Blue No. 4, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, and D&C Yellow No. 7.

23. The physically stable pharmaceutical suspension of claim 1 wherein the active pharmaceutical component is present in an amount of from about 0.1 weight percent to 20.0 weight percent based on the volume of the physically stable pharmaceutical suspension.

24. The physically stable pharmaceutical suspension of claim 1 wherein the water soluble, low viscosity grade cellulose polymer is present in an amount of from about 0.1 weight percent to 5.0 weight percent based on the volume of the physically stable pharmaceutical suspension.

25. The stable pharmaceutical suspension of claim 1 wherein the suspending agent is present in an amount of from about 0.05 weight percent to 5.0 weight percent based on the volume of the stable pharmaceutical suspension.

26. The physically stable pharmaceutical suspension of claim 1 which has a viscosity range of 1000 mPa·s to 2500 mPa·s when measured at 20° C.

27. The physically stable pharmaceutical suspension of claim 1 wherein the active pharmaceutical component which is sparingly soluble to insoluble in water comprises acetaminophen, ibuprofen, guaifenesin or combinations thereof;
wherein the water soluble, low viscosity grade cellulose polymer comprises hydroxypropyl methyl cellulose which forms liquid colloidal solutions having a viscosity of about 6 mPa·s when measured on a 2% w/v aqueous solution at 20° C.; and wherein the suspending agent comprises a water soluble, high viscosity grade cellulose, a polysaccharide, a gum, an acrylic acid derivative or combinations thereof which forms a liquid colloidal solution has a viscosity of from in excess of 50 mPa·s in a 1% w/v aqueous solution.

28. A method of forming a physically stable pharmaceutical suspension comprising:
I) combining
a) an active pharmaceutical component which is sparingly soluble to insoluble in water, wherein the active pharmaceutical component comprises particle have an average particle size in the range of from about 10 μm to about 200 μm;
b) a water soluble, low viscosity grade cellulose polymer with a viscosity range of from about 3 to about 50 mPa·s, measured at 2.0% concentration in water at 20° C.;
c) a suspending agent; and
d) water;
II) dispersing the cellulose polymer and suspending agent in a non-solvent medium followed by hydration with the water, and then incorporating the active pharmaceutical component with high shear mixing.
